(19) United States
(12) Patent Application Publication
(10) Pub. No.: US 2022/0378467 A1
(43) Pub. Date: Dec. 1, 2022

(54) TISSUE TYPE DETECTING MEDICAL DEVICES

(71) Applicant: NOVASCAN, INC., Chicago, IL (US)

(72) Inventors: Les BOGDANOWICZ, Park Ridge, IL (US); Donato M. CERES, Chicago, IL (US); Alexander GRYCUK, Mt. Prospect, IL (US); Daniel Steven GEHRKE, Chicago, IL (US)

(21) Appl. No.: 17/819,563

(22) Filed: Aug. 12, 2022

Related U.S. Application Data
(63) Continuation-in-part of application No. 17/695,748, filed on Mar. 15, 2022.
Application No. 17/695,748 is a continuation-in-part of application No. 17/397,896, filed on Aug. 9, 2021.
Application No. 17/695,748 is a continuation-in-part of application No. 17/412,973, filed on Aug. 26, 2021.
Application No. 17/412,973 is a continuation-in-part of application No. 17/695,745, filed on Mar. 15, 2022.
Application No. 17/695,745 is a continuation-in-part of application No. 17/397,896, filed on Aug. 9, 2021.
Application No. 17/695,745 is a continuation-in-part of application No. 17/412,973, filed on Aug. 26, 2021.
(60) Provisional application No. 63/142,242, filed on Jan. 27, 2021.
(60) Provisional application No. 63/142,247, filed on Jan. 27, 2021.
(60) Provisional application No. 63/142,254, filed on Jan. 27, 2021.
(60) Provisional application No. 63/142,260, filed on Jan. 27, 2021.
(60) Provisional application No. 63/142,242, filed on Jan. 27, 2021.
(60) Provisional application No. 63/142,247, filed on Jan. 27, 2021.
(60) Provisional application No. 63/142,254, filed on Jan. 27, 2021.
(60) Provisional application No. 63/142,260, filed on Jan. 27, 2021.
(60) Provisional application No. 63/142,242, filed on Jan. 27, 2021.
(60) Provisional application No. 63/142,247, filed on Jan. 27, 2021.
(60) Provisional application No. 63/142,254, filed on Jan. 27, 2021.
(60) Provisional application No. 63/142,260, filed on Jan. 27, 2021.
(60) Provisional application No. 63/142,242, filed on Jan. 27, 2021.
(60) Provisional application No. 63/142,247, filed on Jan. 27, 2021.
(60) Provisional application No. 63/142,254, filed on Jan. 27, 2021.
(60) Provisional application No. 63/142,260, filed on Jan. 27, 2021.

(51) Int. Cl. A61B 17/34; A61B 5/0538; A61B 5/00

(52) U.S. Cl. CPC A61B 17/3403; A61B 17/3421; A61B 5/0538; A61B 5/7221; A61B 2017/00026

(57) ABSTRACT

In various embodiments, a medical device comprises a trocar including an awl and a cannula; two or more electrodes disposed on a distal portion of the trocar; an impedance bridge coupled to the two or more electrodes; and a processor coupled to the impedance bridge. In various embodiments, a computer-implemented method for evaluating tissue of a patient comprises recording, at one or more frequencies, one or more impedance measurements, wherein each impedance measurement is associated with two or more electrodes disposed on a distal portion of a trocar; comparing the one or more impedance measurements to one or more characteristic impedances associated with one or more tissue types; and determining, based on the one or more impedance measurements and the one or more characteristic impedances, one or more tissue types at a location associated with the distal portion of the trocar.

### CROSS-REFERENCES TO RELATED APPLICATIONS

The present application is a continuation-in-part of the U.S. patent application titled, “MEDICAL DEVICES CONFIGURED WITH NEEDLE ELECTRODES,” filed on Mar. 15, 2022, and having Ser. No. 17/695,748, which is a continuation-in-part of the U.S. patent application titled, “IMPEDANCE-CALIBRATED DIAGNOSTIC MEDICAL DEVICES,” filed on Aug. 9, 2021, and having Ser. No. 17/397,896, which claims the benefit of U.S. Provisional Patent Application No. 63/142,242, filed Jan. 27, 2021; U.S. Provisional Patent Application No. 63/142,247, filed Jan. 27, 2021; U.S. Provisional Patent Application No. 63/142,254, filed Jan. 27, 2021; and U.S. Provisional Patent Application No. 63/142,260, filed Jan. 27, 2021, and which is also a continuation-in-part of the U.S. patent application titled, “TECHNIQUES FOR CONTROLLING MEDICAL DEVICE TOOLS,” filed on Aug. 26, 2021, and having Ser. No. 17/412,973, which claims the benefit of U.S. Provisional Patent Application No. 63/142,242, filed Jan. 27, 2021; U.S. Provisional Patent Application No. 63/142,247, filed Jan. 27, 2021; U.S. Provisional Patent Application No. 63/142,254, filed Jan. 27, 2021; and U.S. Provisional Patent Application No. 63/142,260, filed Jan. 27, 2021. The present application is also a continuation-in-part of the U.S. patent application titled, “TECHNIQUES FOR DETERMINING TISSUE TYPES,” filed on Mar. 15, 2022, and having Ser. No. 17/695,745, which is a continuation-in-part of the U.S. patent application titled, “IMPEDANCE-CALIBRATED DIAGNOSTIC MEDICAL DEVICES,” filed on Aug. 9, 2021, and having Ser. No. 17/397,896, which claims the benefit of U.S. Provisional Patent Application No. 63/142,242, filed Jan. 27, 2021; U.S. Provisional Patent Application No. 63/142,247, filed Jan. 27, 2021; U.S. Provisional Patent Application No. 63/142,254, filed Jan. 27, 2021; and U.S. Provisional Patent Application No. 63/142,260, filed Jan. 27, 2021, and which is also a continuation-in-part of the U.S. patent application titled, “TECHNIQUES FOR CONTROLLING MEDICAL DEVICE TOOLS,” filed on Aug. 26, 2021, and having Ser. No. 17/412,973, which claims the benefit of U.S. Provisional Patent Application No. 63/142,242, filed Jan. 27, 2021; U.S. Provisional Patent Application No. 63/142,247, filed Jan. 27, 2021; U.S. Provisional Patent Application No. 63/142,254, filed Jan. 27, 2021; and U.S. Provisional Patent Application No. 63/142,260, filed Jan. 27, 2021. The subject matter of these related applications is hereby incorporated herein by reference.

### BACKGROUND

### Field of the Various Embodiments

Embodiments of the present disclosure relate generally to electronics and medical diagnostic technology and, more specifically, to tissue type detecting medical devices.

### Description of the Related Art

In many minimally invasive surgical procedures, a healthcare professional inserts an instrument into a small opening cut into a patient's body to inspect a particular tissue type, such as a tumor or a cyst. For the inspection steps, the healthcare professional oftentimes uses a trocar that includes an awl and a cannula. The healthcare professional first inserts the awl to penetrate the tissue type. The healthcare professional then slides the cannula down the length of the awl. The healthcare professional subsequently removes the awl, leaving the cannula in place. Once setup, the healthcare professional can introduce various instruments through the cannula to perform the relevant surgical procedure. After the surgical procedure, the healthcare professional can leave the cannula in place in order to introduce medication at the location of the procedure or to drain fluid or gas from the body of the patient.

One drawback of using a trocar for surgical procedures is the difficulty of positioning the tip of the cannula at a desired location, such as at the location of a tumor or a cyst. If the cannula is not positioned at the desired location, then the healthcare professional could wind up performing the surgical procedure on tissue at a different, incorrect location, such as at a location where healthy tissue resides instead of on tissue associated with the tumor or cyst. Also, the healthcare professional could determine that the trocar is incorrectly positioned and attempt to reposition the trocar in order to adjust the position of the tip of the cannula. Such an adjustment could cause additional damage to the tissue of the patient. Another drawback is the limited number of instruments that can be concurrently used during a surgical procedure due to the small size of the cannula. For example, during a given surgical procedure, the healthcare professional could attempt to use a light source, a camera, a diagnostic probe, and a cutting tool concurrently, which could be difficult due to the limited space available at the location associated with the distal portion of the trocar. Also, the diameter of the cannula may not be large enough to accommodate all of the instruments that a medical professional wants to use concurrently during a given surgical procedure.

As the foregoing illustrates, what is needed in the art are more effective ways to detect tissue types using medical devices.

### SUMMARY

Embodiments are disclosed for medical devices. In various embodiments, a medical device comprises a trocar including an awl and a cannula; two or more electrodes disposed on a distal portion of the trocar; an impedance bridge coupled to the two or more electrodes; and a processor coupled to the impedance bridge.

In various embodiments, a computer-implemented method for evaluating tissue of a patient comprises recording, at one or more frequencies, one or more impedance measurements, wherein each impedance measurement is associated with two or more electrodes disposed on a distal portion of a trocar; comparing the one or more impedance measurements to one or more characteristic impedances associated with one or more tissue types; and determining, based on the one or more impedance measurements and the one or more characteristic impedances, one or more tissue types at a location associated with the distal portion of the trocar.

At least one technical advantage of the disclosed medical device relative to the prior art is that the disclosed medical device is able to automatically and accurately determine the tissue type at a location associated with the distal portion of the trocar during a surgical procedure. For example, the disclosed trocar can accurately determine whether the tissue type at a location associated with the distal portion of the trocar is a tumor tissue type, a cyst tissue type, or a non-tumor and non-cyst tissue type. Determining the tissue type at the location associated with the distal portion of the trocar with high accuracy can advantageously reduce the incidence of false positive testing outcomes and reduce the occurrence of unnecessary medical procedures. Determining the tissue type of the tissue associated with the distal portion of the trocar with high accuracy also can advantageously reduce the incidence of false negative testing outcomes and enable effective cancer treatment at early stages and with better prognoses. In addition, the disclosed medical device can automatically and accurately determine whether the tissue type at the location associated with the distal portion of the trocar matches an expected tissue type at the location of a given surgical procedure. Such determinations can improve overall confidence in the test results. Likewise, automatically and accurately determining that the tissue type at a location associated distal portion of the trocar does not match the expected tissue type can reduce incorrect test results and give a healthcare professional an opportunity to reposition the trocar. These technical advantages provide one or more technological advancements over prior art designs and approaches.

### BRIEF DESCRIPTION OF THE DRAWINGS

FIG. 1 illustrates a medical device, according to various embodiments;

FIG. 2 is an illustration of the trocar of FIG. 1, according to various embodiments;

FIG. 3 is another illustration of the trocar of FIG. 1, according to other various embodiments;

FIG. 4 is a more detailed illustration of the distal portion of the cannula of FIG. 3, according to various embodiments;

FIG. 5 is another illustration of the trocar of FIG. 1, according to other various embodiments;

FIG. 6 is a more detailed illustration of the distal portion of the trocar of FIG. 5, according to various embodiments;

FIG. 7 is an illustration of the external electrical components of FIG. 1, according to various embodiments;

FIG. 8 is a more detailed illustration of the medical device of FIG. 1, according to various embodiments;

FIG. 9 is a more detailed illustration of the medical device of FIG. 1, according to other various embodiments;

FIG. 10 more detailed illustration of the medical device of FIG. 1, according to other various embodiments; and

FIG. 11 is a flow diagram of method steps for determining one or more tissue types at a location associated with a distal portion of a trocar, according to various embodiments.

### DETAILED DESCRIPTION

In the following description, numerous specific details are set forth to provide a more thorough understanding of the various embodiments. However, in the range of embodiments of the concepts includes some embodiments omitting one or more of these specific details.

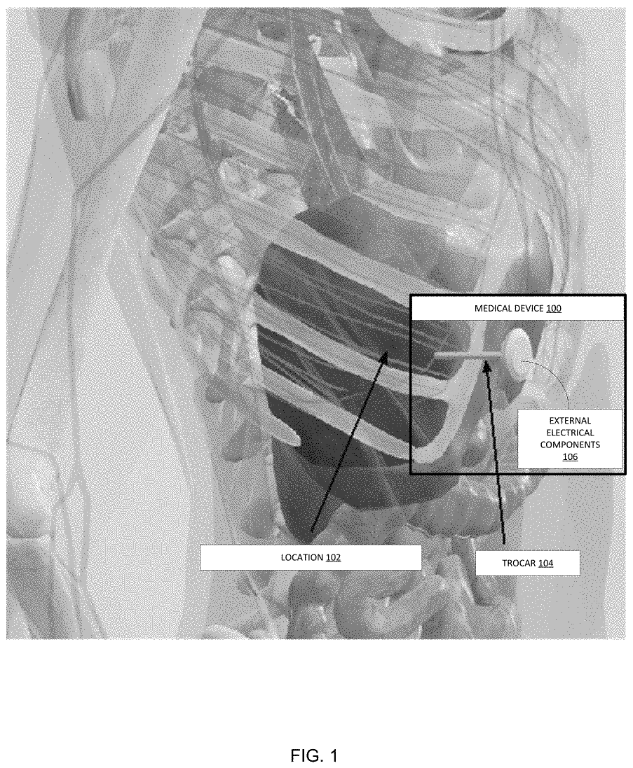

FIG. 1 illustrates a medical device, according to various embodiments. As shown, the medical device 100 includes, without limitation, a trocar 104 and external electrical components 106.

The trocar 104 is inserted into a body of a patient. While not shown, the trocar 104 includes an awl and a cannula. The awl is inserted to penetrate the tissue of the body of the patient. The cannula slides down the length of the awl and is positioned at a location 102 in the body of the patient, such as a location of a tumor or a cyst. The awl is removed, leaving the cannula in place at the location 102. Two or more electrodes are disposed on a distal portion of the trocar 104 (including, without limitation, a distal portion of the awl and/or a distal portion of the cannula). The external electrical components 106 are coupled to the electrodes disposed on the distal portion of the trocar 104 by wires. The external electrical components 106 generate current between the two or more electrodes at one or more frequencies. The external electrical components 110 include a processor that records one or more impedance measurements of the current conducted through tissue between at least two of the two or more electrodes. As described in greater detail below, the medical device 100 determines, based on the impedance measurements at the one or more frequencies, one or more tissue types at the location associated with the distal portion of the trocar 104.

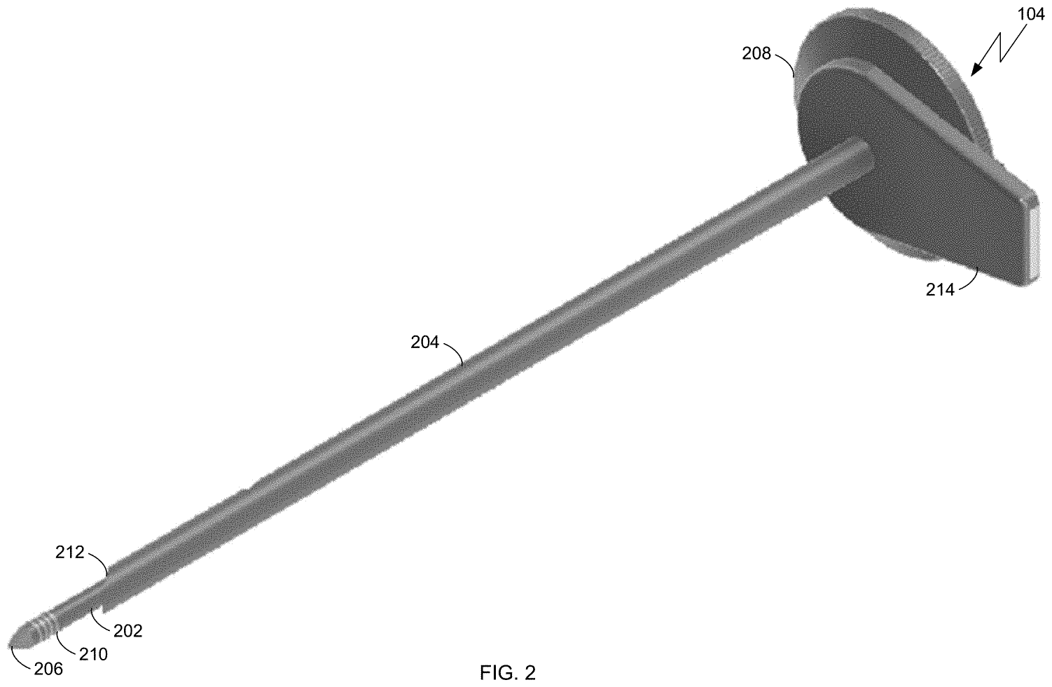

FIG. 2 is an illustration of the trocar 104 of FIG. 1, according to various embodiments. As shown, the trocar 104 includes an awl 202 and a cannula 204. The awl 202 includes a distal portion 206 terminating in a tip, a handle 208, and two or more electrodes 210 disposed on the distal portion 206 of the awl 202. The cannula 204 includes a handle 214 and a distal portion 212.

As shown, each of the two or more electrodes 210 is disposed on an outer surface of the distal portion 206 of the awl 202. More specifically, each of the two or more electrodes 210 is disposed at a respective location along a length of the distal portion 206 of the awl 202. While not shown, the two or more electrodes 210 disposed on the distal portion 206 of the awl are coupled to external electrical components 106 by wires. In various embodiments, at least a portion of the wires are imprinted on an outer surface of the awl 202. The external electrical components 106 can generate current at one or more frequencies that is conducted between at least two of the two or more electrodes 210. The external electrical components 106 can record impedance measurements while the current is conducted between at least two of the two or more electrodes 210.

In various embodiments, the external electrical components 106 (not shown) record impedance measurements between different pairs of the two or more electrodes 210, such as a first impedance measurement of current conducted between a first electrode 210 and a second electrode 210 of the two or more electrodes 210 and a second impedance measurement of current conducted between the second electrode 210 and a third electrode 210 of the two or more electrodes 210. Recording impedance measurements for different pairs of the electrodes 210 while the awl 202 is inserted into a body of a patient can indicate different tissue types of tissue at different portions the location associated with the distal portion 206 of the awl 202, such as tissue types at different depths of the location 102 associated with the distal portion 206 of the awl 202.

While not shown, the various embodiments discussed in relation to FIG. 2, in which two or more electrodes 210 are disposed at respective locations along a length of the awl 202 could be adapted for embodiments in which the two or more electrodes 210 are disposed at respective locations along a length of the cannula 204. For example, in other various embodiments, two or more electrodes 210 could be disposed at respective lengths along the distal portion 212 of the cannula 204. At least some of the features of various embodiments as discussed in relation to FIG. 2 would similarly apply to other various embodiments in which the two or more electrodes 210 are disposed at respective lengths along the distal portion 212 of the cannula 204.

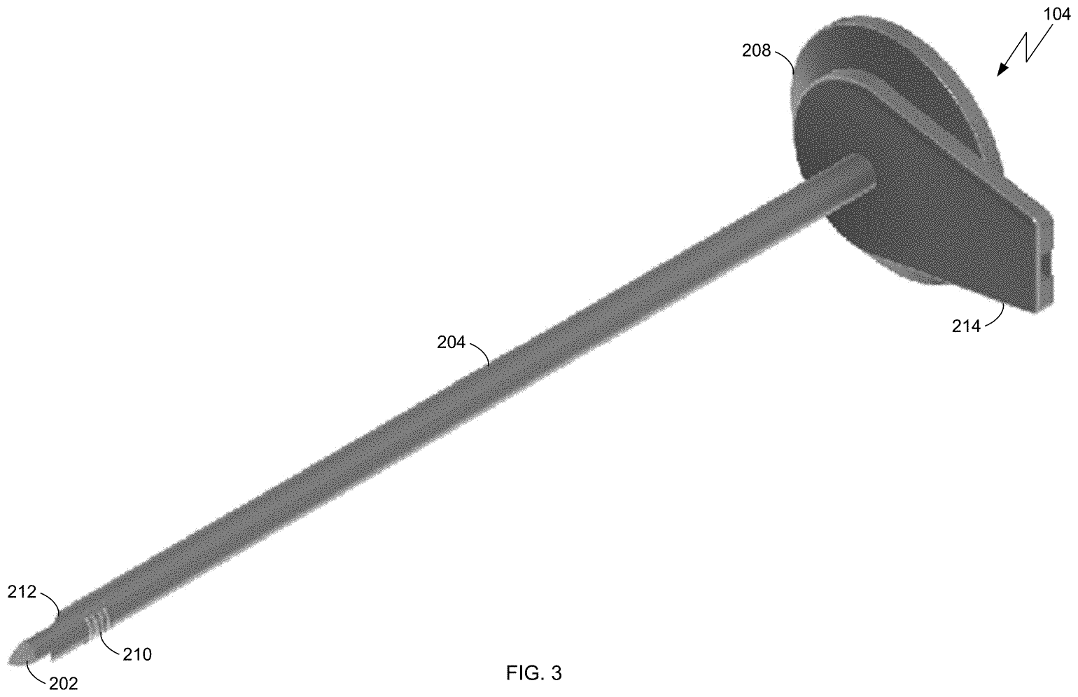

FIG. 3 is another illustration of the trocar of FIG. 1, according to other various embodiments. As shown, the trocar 104 includes an awl 202 and a cannula 204. The awl 202 includes a handle 208. The cannula 204 includes a handle 214, a distal portion 212, and two or more electrodes 210 disposed on the distal portion 206 of the cannula 204.

As shown, each of the two or more electrodes 210 is disposed on an outer surface of the distal portion 212 of the cannula 204. More specifically, each of the two or more electrodes 210 is disposed at a respective location along a length of the distal portion 212 of the cannula 204, and also on one side of the cannula 204. The cannula 204 can slide along a length of the awl 202. While not shown, the two or more electrodes 210 disposed on the distal portion 212 of the cannula 204 are coupled to external electrical components 106 by wires. In various embodiments, at least a portion of the wires are imprinted on an outer surface of the cannula 204. The external electrical components 106 can generate current at one or more frequencies that is conducted between at least two of the two or more electrodes 210.

In various embodiments, the external electrical components 106 (not shown) record impedance measurements between different pairs of the two or more electrodes 210, such as a first impedance measurement of current conducted between a first electrode 210 and a second electrode 210 of the two or more electrodes 210 and a second impedance measurement of current conducted between the second electrode 210 and a third electrode 210 of the two or more electrodes 210. Impedance measurements for different pairs of the electrodes 210 can indicate different tissue types of tissue at different locations along the length of the cannula 204 and on the one side of the cannula 204, such as tissue types of tissue at different depths of the location 102 associated with the distal portion 212 of the cannula 204 and on the one side of the cannula 204.

In various embodiments, the cannula 204 slides along a length of the awl 202. When the awl is inserted into the body of the patient, the cannula 204 can be positioned at different locations along the length of the awl 202. Different positions of the cannula 204 cause the distal portion 212 of the cannula 204 to be positioned at different locations within the body of the patient, such as different depths within the body of the patient. In various embodiments, the external electrical components 106 record impedance measurements between the two or more electrodes 210 while the cannula 204 is located at different locations along the length of the awl 202, such as a first impedance measurement associated with a first location of the cannula 204 along the length of the awl 202 and a second impedance measurement associated with a second location of the cannula 204 along the length of the awl 202. Impedance measurements for different locations of the cannula 204 along the length of the awl 202 can indicate different tissue types of tissue at different locations associated with the distal portion 212 of the cannula 204, such as tissue types of tissue at different depths along the length of the awl 202 and on the one side of the cannula 204 on which the two or more electrodes 210 are disposed.

As shown, the two or more electrodes 210 are disposed on one side of the cannula 204. When the cannula 204 is located within the body of the patient at a first angle of rotation in which the two or more electrodes 210 contact tissue on one side of the awl 202, such as a left side of the awl 202. The cannula 204 can be rotated to a second angle of rotation in which the two or more electrodes 210 contact tissue on another side of the awl 202, such as a right side of the awl 202. In various embodiments, the external electrical components 106 record impedance measurements between the two or more electrodes 210 when the cannula is rotated to different angles of rotation. Impedance measurements for different angles of rotation of the cannula 204 can indicate different tissue types of tissue at different locations the length of the cannula 204 and on the one side of the cannula 204, such as tissue types on different sides of the awl 202. In various embodiments, impedance measurements can be recorded for various combinations of locations of the cannula 204 along the awl 202 and angles of rotation. The impedance measurements can indicate different tissue types of tissue at different depths along the length of the awl 202 and on different sides of the awl 202.

In various embodiments, the cannula 204 remains in the body of the patient after the awl 202 is removed. For example (without limitation), the cannula 204 can be affixed to maintain the distal portion 212 of the cannula 204 at a location 102 within the body of the patient. The cannula 204 can introduce medication at the location 102 associated with the distal portion 212 of the cannula 204 and/or drain fluid or gas from the body of the patient. In various embodiments, the external electrical components 106 record impedance measurements between the two or more electrodes 210 at different times, such as on different days of treatment of a tumor or a cyst. The impedance measurements recorded at different times can indicate a progression of treatment of tissue at the location 102 associated with the distal portion 212 of the cannula 204, such as (without limitation) a size of a tumor or cyst at the location 102 and/or a tissue type of tissue at the location 102. Alternatively or additionally, the impedance measurements can determine a position of the distal portion 212 of the cannula 204. For example (without limitation), impedance measurements at different times that are consistent with one another can indicate that the distal portion 212 of the cannula 204 remains correctly positioned at the location 102. Impedance measurements at different times that are inconsistent with one another can indicate that the distal portion 212 of the cannula 204 has been displaced or dislodged from the location 102, which can alert a healthcare professional to reposition the distal portion 212 of the cannula 204.

While not shown, in various embodiments, at least one electrode 210 of the two or more electrodes 210 can be disposed on an inner surface of the cannula 204. More specifically, at least one of the two or more electrodes 210 is disposed at a respective location along a length of the inner surface of the distal portion 212 of the cannula 204. In various embodiments, one or more electrodes 210 are disposed on an outer surface of the distal portion 212 of the cannula 204, and another one or more electrodes 210 are disposed on an inner surface of the distal portion 212 of the cannula 204. Disposing at least one of the electrodes 210 on the inner surface of the cannula 204 can protect the electrodes 210 from contact with the tissue or medical equipment while the cannula 204 slides down the awl 202. Alternatively or additionally, disposing at least two of the electrodes 210 on the inner surface of the cannula 204 can enable the external electrical components 106 to record impedance measurements of tissue within the cannula, such as a tissue sample extracted by a tissue sample extraction tool.

While not shown, the various embodiments discussed in relation to FIG. 3, in which two or more electrodes 210 are disposed on one or more sides of the cannula 204, could be adapted for embodiments in which the two or more electrodes 210 are disposed on one or more sides of the awl 202. For example, in other various embodiments, two or more electrodes 210 could be disposed on one side of the distal portion 206 of the awl 202. At least some of the features of various embodiments as discussed in relation to FIG. 3 would similarly apply to other various embodiments in which the two or more electrodes 210 are disposed on one side of the distal portion 206 of the awl 202.

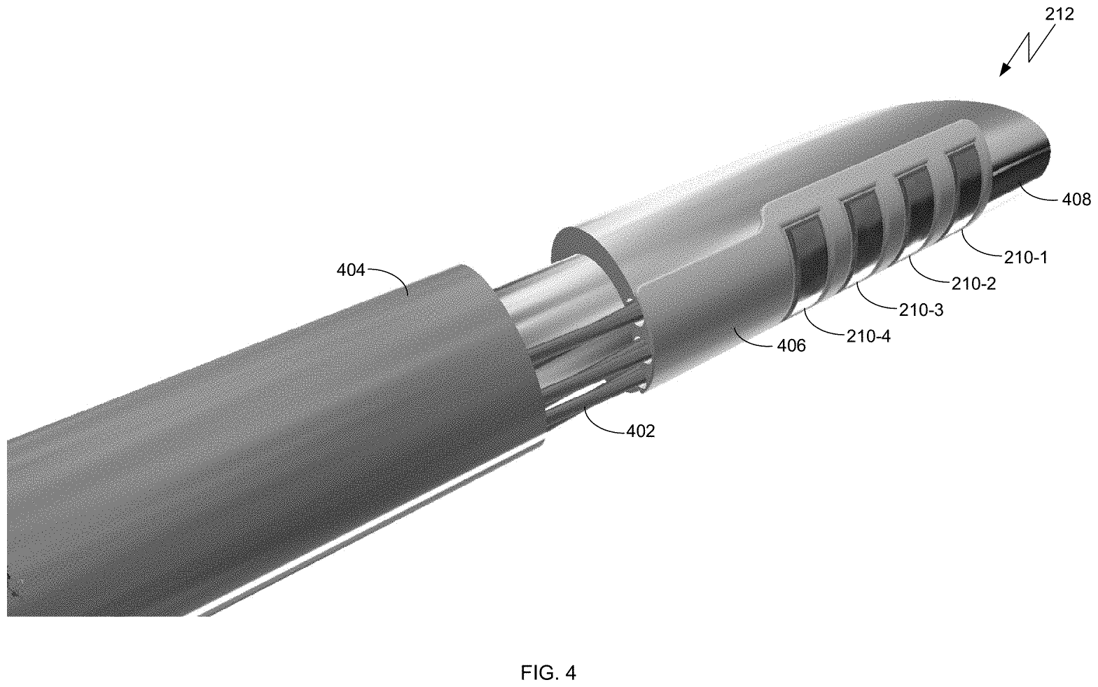

FIG. 4 is a more detailed illustration of the distal portion 212 of the cannula 204 of FIG. 3, according to various embodiments. As shown, the distal portion 212 of the cannula 204 includes a set of electrodes 210-1 to 210-4, a conduit 404, electrically insulating material 406, and an aperture 408. As shown, the conduit 404 includes wires 402.

In various embodiments, the electrically insulating material 406 is located between adjacent pairs of electrodes 210 along the length of the distal portion 212 of the cannula 204. As shown, the electrically insulating material 406 includes a set of carve-outs, and each of the two or more electrodes 210 is located within one of the carve-outs of the electrically insulating material 406. The electrically insulating material 406 can reduce contact and short circuits between adjacent electrodes 210, which could reduce the accuracy of the impedance measurements. While not shown, the two or more electrodes 210 disposed on the distal portion 206 of the cannula 204 are coupled to external electrical components 106 by the wires 402. The external electrical components 106 can generate current at one or more frequencies that is conducted between at least two of the two or more electrodes 210. The external electrical components 106 can record impedance measurements while the current is conducted between at least two of the two or more electrodes 210.

In various embodiments, the external electrical components 106 are selectively coupled to two or more selected electrodes 210 of the two or more electrodes 210. While the external electrical components 106 are selectively coupled to a first subset of the two or more electrodes 210 (e.g., without limitation, the first electrode 210-1 and the second electrode 210-2), the medical device can record a first subset of impedance measurements of tissue between or contacting the first subset of the two or more electrodes 210. While the external electrical components 106 are selectively coupled to a second subset of the two or more electrodes 210 (e.g., without limitation, the third electrode 210-3 and the fourth electrode 210-4), the medical device can record a second subset of impedance measurements of tissue between or contacting the second subset of the two or more electrodes 210. Based on the first subset and second subset of impedance measurements, the medical device can determine a first tissue type between the first electrode 210-1 and the second electrode 210-2 and a second tissue type between the third electrode 210-3 and the fourth electrode 210-4. For example and without limitation, the first tissue type based on the first impedance measurement can indicate that the tissue near the aperture 408 of the cannula 204 is a tumor tissue type, and the second tissue type based on the second impedance measurement can indicate that the tissue further from the aperture 408 is a non-tumor tissue type. As another example and without limitation, the first tissue type based on the first impedance measurement can indicate that the tissue near the aperture 408 a cyst tissue type, and the second tissue type based on the second impedance measurement can indicate that the tissue further from the aperture 408 is a non-cyst tissue type. Based on the first and second tissue types, the external electrical components 106 can determine the tissue type of tissue associated with the distal portion 212 of the cannula 204 and/or whether the distal portion 212 of the cannula 204 is positioned at the location 102.

While not shown, in various embodiments, one or more of the two or more electrodes 210 is located on a first side of the distal portion 212 of the cannula 204, and at least another one or more of the two or more electrodes 210 is located on a second side of the distal portion 212 of the cannula 204. For example and without limitation, the first electrode 210-1 and the second electrode 210-2 could be located on a left side of the distal portion 212 of the cannula 204. The external electrical components 106 could selectably couple to a first subset of the two or more electrodes 210 (e.g., without limitation, the first electrode 210-1 and the second electrode 210-2) to record a first subset of impedance measurements. Based on the first subset of impedance measurements, the external electrical components 106 could determine a tissue type on the left side of the distal portion 212 of the cannula 204. Also, the third electrode 210-3 and the fourth electrode 210-4 could be located on a right side of the distal portion 212 of the cannula 204. The external electrical components 106 could selectably couple to a second subset of the two or more electrodes 210 (e.g., without limitation, the third electrode 210-3 and the fourth electrode 210-4) to record a second subset of impedance measurements. Based on the second subset of impedance measurements, the external electrical components 106 could determine a tissue type on the right side of the distal portion 212 of the cannula 204. Based on the first and second subsets of impedance measurements, the external electrical components 106 could determine the tissue types on different sides of the distal portion 212 of the cannula 204. As an example and without limitation, the external electrical components 106 could determine that the tissue on a left side of the cannula 204 is a tumor tissue type, and that the tissue on a right side of the cannula 24 is a non-tumor tissue type. As another example and without limitation, the external electrical components 106 could determine that the tissue on a left side of the cannula 204 is a cyst tissue type, and that the tissue on a right side of the cannula 24 is a non-cyst tissue type.

While not shown, the various embodiments discussed in relation to FIG. 4, in which the two or more electrodes 210 are disposed on one or more sides of the cannula 204, could be adapted for embodiments in which the two or more electrodes 210 are disposed on one or more sides of the awl 202. For example and without limitation, in other various embodiments, the awl 202 could include a conduit 404 and electrically insulating material 406, and the two or more electrodes 210 could be disposed on one side of the distal portion 206 of the awl 202. At least some of the features of various embodiments as discussed in relation to FIG. 4 would similarly apply to other various embodiments in which the two or more electrodes 210 are disposed on one side of the distal portion 206 of the awl 202.

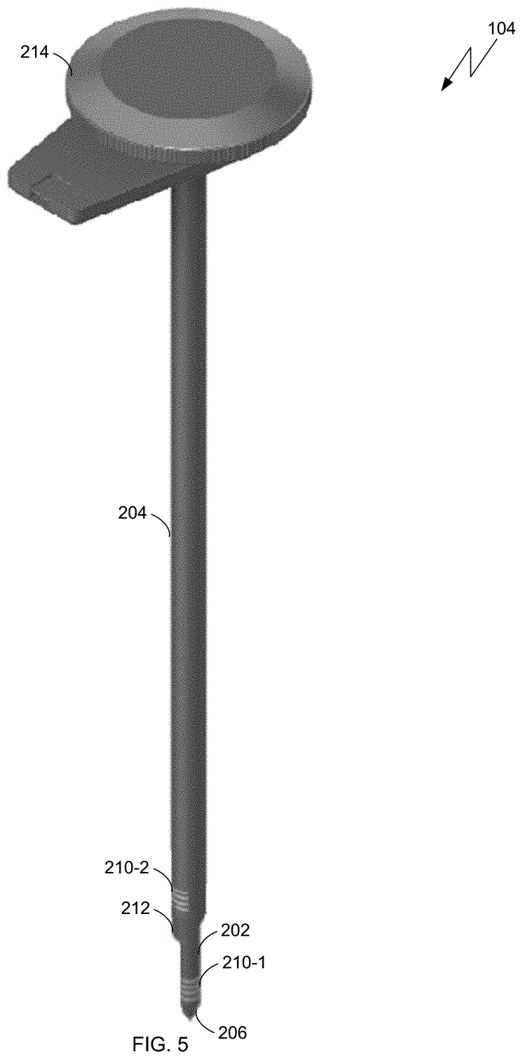

FIG. 5 is another illustration of the trocar of FIG. 1, according to other various embodiments. As shown, the trocar 104 includes an awl 202 and a cannula 204. The awl 202 includes a distal portion 206 terminating in a tip, a handle 208, and a first set of one or more electrodes 210-1 disposed on the distal portion 206 of the awl 202. The cannula 204 includes a handle 214, a distal portion 212 including an aperture, and a second set of one or more electrodes 210-2 disposed on the distal portion 206 of the cannula 204.

As shown, each of the one or more electrodes 210 in the first set of one or more electrodes 210-1 is disposed at a respective location along a length of the distal portion 206 of the awl 202. Also, each of the one or more electrodes 210 in the second set of one or more electrodes 210-2 is disposed at a respective location along a length of the distal portion 212 of the cannula 204. While not shown, both the one or more electrodes 210 of the first set of electrodes 210-1 and the one or more electrodes 210 of the second set of electrodes 210-2 are coupled to external electrical components 106 by wires 402. The external electrical components 106 can generate current at one or more frequencies that is conducted between at least two electrodes 210 of two or more electrodes 210 of the first set of electrodes 210-1. Alternatively or additionally, the external electrical components 106 can generate current at one or more frequencies that is conducted between at least two electrodes 210 of two or more electrodes 210 of the second set of electrodes 210-2. Alternatively or additionally, the external electrical components 106 can generate current at one or more frequencies that is conducted between at least one of the one or more electrodes 210 of the first set of electrodes 210-1 and at least one of the one or more electrodes 210 of the second set of electrodes 210-2.

In various embodiments, the cannula 204 slides along a length of the awl 202. When the awl is inserted into the body of the patient, the cannula 204 can be positioned at different locations along the length of the awl 202. Different positions of the cannula 204 cause the distal portion 212 of the cannula 204 to be positioned at different locations within the body of the patient, such as different depths within the body of the patient. In various embodiments, the external electrical components 106 record impedance measurements between at least two electrodes 210 while the cannula 204 is located at different locations along the length of the awl 202, wherein each electrode 210 of the at least two electrodes 210 is included either in the first set of electrodes 210-1 disposed on the distal portion 206 of the awl 202 or in the second set of electrodes 210-2 disposed on the distal portion 212 of the cannula 204. For example and without limitation, the impedance measurements can include a first impedance measurement associated with a first location of the cannula 204 along the length of the awl 202 and a second impedance measurement associated with a second location of the cannula 204 along the length of the awl 202. Impedance measurements for different locations of the cannula 204 along the length of the awl 202 can indicate different tissue types of tissue at different locations associated with the distal portion 212 of the cannula 204, such as tissue types of tissue at different depths along the length of the awl 202.

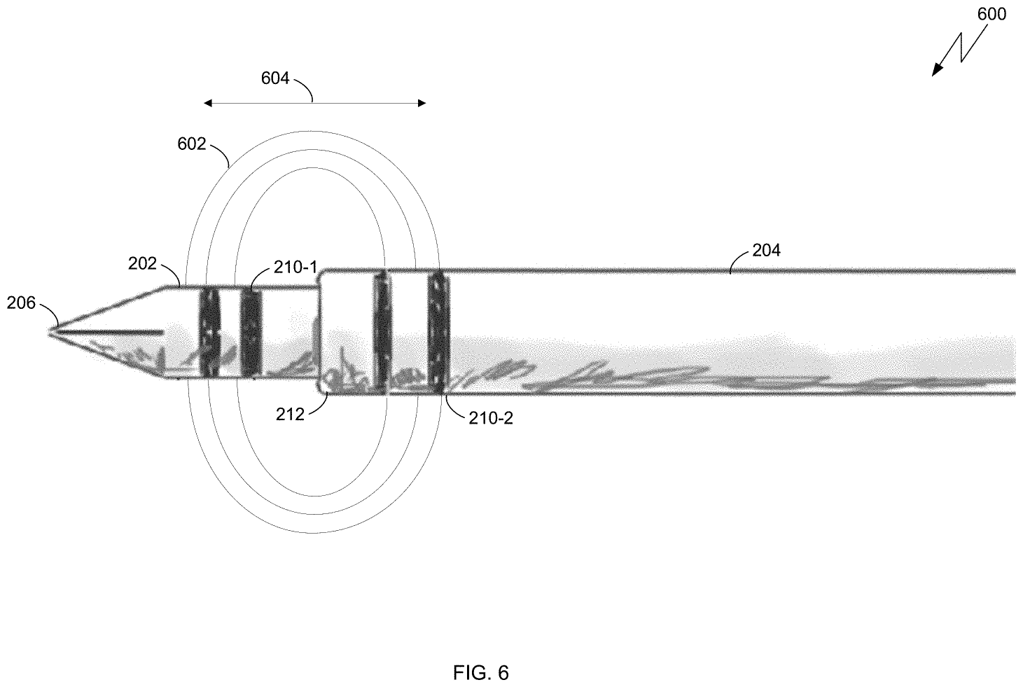

FIG. 6 is a more detailed illustration of the distal portion 600 of the trocar 104 of FIG. 5, according to various embodiments. As shown, the distal portion 600 includes a first set of one or more electrodes 210-1 disposed on a distal portion 206 of the awl 202 and a second set of one or more electrodes 210-2 disposed on a distal portion 212 of the cannula 204. While not shown, the distal portion 600 of the trocar 104 includes an electromagnetic field sensor.

As shown, when the external electrical components 106 conduct current between at least one of the one or more electrodes 210 of the first set of one or more electrodes 210-1 and at least one of the one or more electrodes 210 of the second set of one or more electrodes 210-2, an electromagnetic field 602 is created near the distal portion 600 of the trocar 104. A magnitude of the electromagnetic field 602 is proportional to a distance 604 between the electrodes 210 of the first set of one or more electrodes 210-1 disposed on the distal portion 206 of the awl 202 and the electrodes 210 of the second set of one or more electrodes 210-2 disposed on the distal portion 212 of the cannula 204. In various embodiments, the electromagnetic field sensor (not shown) includes one or more electric field sensors, such as (without limitation) an optical electric field sensor or the like, and/or one or more magnetic field sensor, such as (without limitation) a reed switch, a Hall effect sensor, or the like. The electromagnetic field sensor can be coupled to the external electrical components 106 by wires 402. The electromagnetic field sensor can record one or more measurements of the magnitude of the electromagnetic field while the external electrical components 106 conduct current between the at least one of the one or more electrodes 210 of the first set of one or more electrodes 210-1 and the at least one of the one or more electrodes 210 of the second set of one or more electrodes 210-2. The one or more measurements of the magnitude of the electromagnetic field can indicate the distance 604 between the distal portion 206 of the awl 202 and the distal portion 212 of the cannula 204. That is, the one or more measurements of the magnitude of the electromagnetic field can indicate the distance of an aperture 408 in the distal portion 212 of the cannula 204 to a tip of the distal portion 206 of the awl 202.

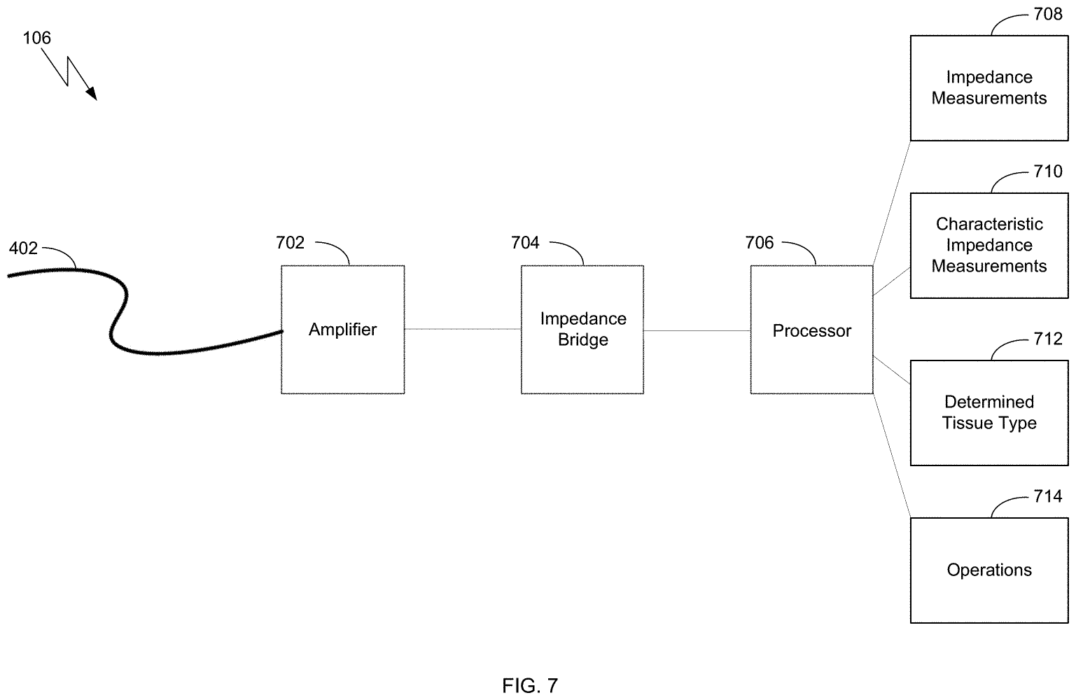

FIG. 7 is an illustration of the external electrical components of FIG. 1, according to various embodiments. As shown, the external electrical components 106 include wires 402, an amplifier 702, an impedance bridge 704, and a processor 706. The wires 402 conduct current at various frequencies between two or more electrodes 210 disposed on a distal portion 600 of the trocar 104 and the external electrical components 106. In various embodiments, the amplifier 702 is an analog interface amplifier that amplifies a supplied voltage and/or a return voltage while the wires 402 conduct current at various frequencies between the impedance bridge 704 and the two or more electrodes 210. In various embodiments, the impedance bridge 704 is an impedance load that the processor 706 measures to determine an impedance of a circuit including the impedance bridge 704, the amplifier 702, and the two or more electrodes 210. The processor 706 generates frequencies for a current that the wires 402 conduct between the impedance bridge 704 and the selected two or more electrodes 210.

While the wires 402 conduct current at various frequencies, the processor 706 records one or more impedance measurements 708 of the circuit including the at least two electrodes 210. The processor 706 compares the one or more impedance measurements 708 with characteristic tissue types 712 of respective one or more tissue types. Based on the one or more impedance measurements 708 and the characteristic tissue types 712, the processor 706 determines one or more tissue types 712 of tissue at the location 102 associated with the distal portion 600 of the trocar 104. For example and without limitation, based on the one or more impedance measurements 708 and the characteristic tissue types 712, the processor 706 can determine which tissue type is associated with characteristic impedance measurements 710 that are closest to the impedance measurements 708 of the portion of tissue between at least two of the two or more electrodes 210. In various embodiments, the processor 706 can determine a Cole relaxation frequency of the portion of tissue based on the impedance measurements 708, and can compare the Cole relaxation frequency to one or more characteristic Cole relaxation frequencies of one or more tissue types. The Cole relaxation frequency corresponds to a frequency associated with a greatest impedance measurement 708 included in the one or more impedance measurements 708. In various embodiments, the Cole relaxation frequency is a frequency of a maximum normalized impedance measurement of the portion of tissue between at least two of the two or more electrodes 210. For example and without limitation, based on a Cole relaxation frequency below a threshold frequency (e.g., 10⁵ Hz), the processor 706 can determine that the portion of tissue between at least two of the two or more electrodes 210 is a non-tumor tissue type. Similarly, for example and without limitation, based on a Cole relaxation frequency above the threshold frequency, the processor 706 can determine that the portion of tissue between at least two of the two or more electrodes 210 is a tumor tissue type. As another example and without limitation, based on a Cole relaxation frequency below a threshold frequency, the processor 706 can determine that the portion of tissue between at least two of the two or more electrodes 210 is a cyst tissue type. Similarly, for example and without limitation, based on a Cole relaxation frequency above the threshold frequency, the processor 706 can determine that the portion of tissue between at least two of the two or more electrodes 210 is a non-cyst tissue type.

In some embodiments, the processor 706 determines two or more tissue types of tissue at the location associated with the distal portion 600 of the trocar 104 based on impedance measurements respectively recorded by different electrode pairs of the two or more electrodes 210. For example and without limitation, a first pair of electrodes 210 can be disposed at a first location along the length of the distal portion 206 of the awl 202 and/or the distal portion 212 of the cannula 204. A second pair of electrode 210 can be disposed at a second location along the length of the distal portion 206 of the awl 202 and/or the distal portion 212 of the cannula 204. While the trocar 104 is inserted into a body of a patient, the processor 706 can determine tissue types at the first location and the second location based on the respective impedance measurements associated with the first pair of electrodes 210 and the second pair of electrodes 210. As another example and without limitation, the distal portion 60 of the trocar 104 can include a first pair of electrodes 210 disposed on a left side of the distal portion 600 and a second pair of electrodes 210 disposed on a right side of the distal portion 600. While the trocar 104 is inserted into a body of a patient, the processor 706 can determine tissue types at the left side and the right side of the distal portion 600 of the trocar 104 based on the respective impedance measurements associated with the first pair of electrodes 210 and the second pair of electrodes 210.

In various embodiments, the external electrical components 106 include a wireless transmitter. For example and without limitation, the wireless transmitter can include a Bluetooth transmitter or a WiFi transmitter. The processor 706 can transmit, using the wireless transmitter, a wireless signal indicating the one or more tissue types at the location associated with a distal portion 600 of the trocar 104. For example and without limitation, the wireless signal can be transmitted to a mobile device of a healthcare professional. Transmitting the wireless signal can cause the mobile device to output a visual indication or an audio indication of the determined tissue type 712.

In various embodiments, the trocar 104 includes one or more medical device tools, and the processor 706 performs one or more operations 714 to control the one or more medical device tools based on the one or more tissue types 712 at the location associated with the distal portion 600 of the trocar 104. For example and without limitation, in various embodiments in which the distal portion 600 of the trocar 104 includes a therapeutic drug delivery tool, the processor 706 can perform operations 714 that include activating the therapeutic drug delivery tool to deliver one or more therapeutic drugs to the tissue at the location 102 associated with the distal portion 600 of the trocar 104. For example and without limitation, in various embodiments in which the distal portion 600 of the trocar 104 includes an energy delivery tool, the processor 706 can perform operations 714 that include activating the energy delivery tool to deliver energy to the tissue at the location 102 associated with the distal portion 600 of the trocar 104. For example and without limitation, in various embodiments in which the distal portion 600 of the trocar 104 includes a tissue sample extraction tool, the processor 706 can perform operations 714 that include activating the tissue sample extraction tool to extract tissue from the location 102 associated with the distal portion 600 of the trocar 104.

In various embodiments, the processor 706 can determine a confidence score of the one or more tissue types at the location associated with the distal portion 600 of the trocar 104. For example and without limitation, the processor 706 can determine a magnitude of difference between the impedance measurements 708 of the electrodes 210 that are associated with a tumor tissue type and the impedance measurements 708 of the electrodes 210 that are associated with a non-tumor tissue type. Alternatively or additionally, the processor 706 can determine a magnitude of difference between the impedance measurements 708 of the electrodes 210 that are associated with a cyst tissue type and the impedance measurements 708 of the electrodes 210 that are associated with a non-cyst tissue type. The confidence score can be based on (e.g., proportional to) the magnitude of difference between the different impedance measurements 708. As another example and without limitation, the processor 706 can determine a magnitude of the difference between the impedance measurements 708 for a set of electrodes 210 and the closest set of characteristic impedance measurements 710 of a tissue type. The confidence score can be based on (e.g., proportional to) the magnitude of the difference between the recorded impedance measurements 708 and the characteristic impedance measurements 710 of the tissue type. As yet another example and without limitation, the processor 706 can determine a signal-to-noise ratio of one or more of the impedance measurements 708. In various embodiments and without limitation, the processor 706 outputs the confidence score along with the determined tissue type 712. The confidence score can indicate to a healthcare professional a confidence of the determined tissue type 712 and/or a confidence of whether the distal portion 600 of the trocar 104 is positioned at the location 102 associated with the surgical procedure.

In various embodiments, the processor 706 can measure one or more dimensions of tissue residing at the location associated with the distal portion 600 of the trocar 104. For example and without limitation, the processor 706 can selectively couple to respective pairs of adjacent electrodes 210 disposed on the distal portion 600 of the trocar 104, wherein each electrode 210 of the pair of adjacent electrodes 210 is disposed on a distal portion 206 of the awl 202 or a distal portion 212 of the cannula 204. The processor 706 can record a subset of impedance measurements 708 for each pair of electrodes 210 disposed on the distal portion 600 of the trocar 104. Based on the tissue types 712 determined for each subset of impedance measurements 708, the processor 706 can determine which pairs of electrodes 210 (e.g., without limitation, which lengthwise portions of the distal portion 600 of the trocar 700) are in contact with a tumor and/or cyst and which pairs of electrodes 210 are not in contact with a tumor and/or cyst. Based on these determinations, the processor 706 can determine a length of a tumor or cyst.

In various embodiments in which the medical device 100 includes an output component, the processor 606 performs one or more operations 614 to output an indication of the one or more tissue types at the location associated with the distal portion 600 of the trocar 104. In various embodiments and without limitation, the medical device 100 can display the one or more tissue types 612 at the location 102 associated with the distal portion 600 of the trocar 104 using a visual output (e.g., without limitation, a liquid crystal display (LCD), a light-emitting diode (LED) display to present a visual indication of the one or more tissue types 612 at the location 102 associated with the distal portion 600 of the trocar 104, such as a light, symbol, text, graphic, or the like). In various embodiments and without limitation, the processor 606 can output a visual indication that the one or more tissue types 612 at the location 102 associated with the distal portion 600 of the trocar 104 is a cyst tissue type. The visual indication can indicate whether or not the distal portion 600 of the trocar 104 is positioned at the location 102 of the surgical procedure.

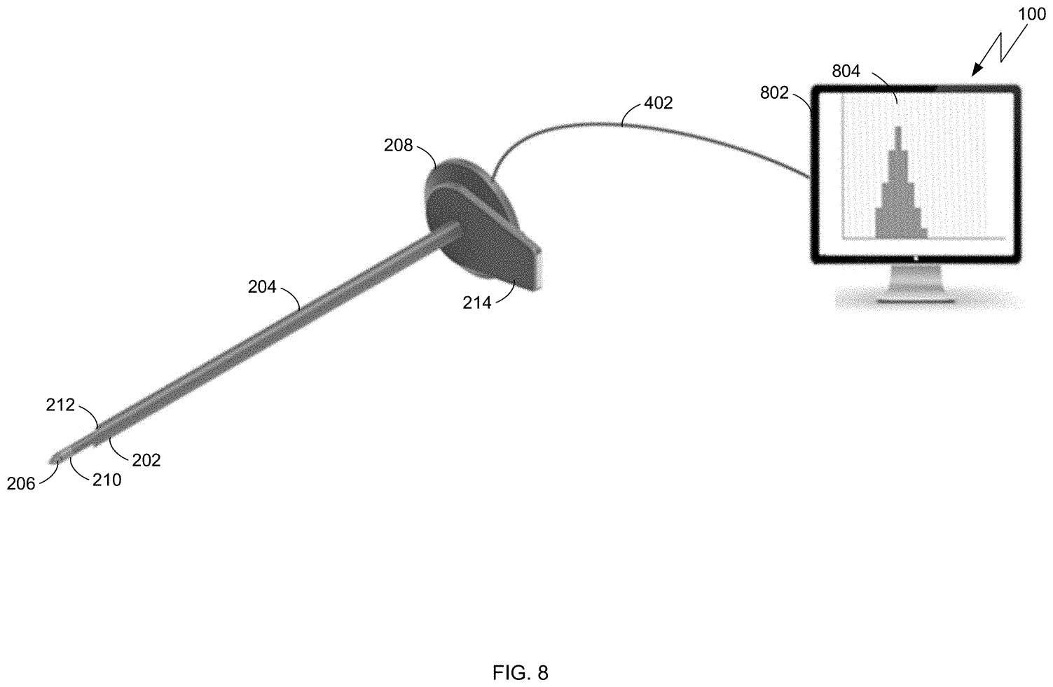

FIG. 8 is a more detailed illustration of the medical device 100 of FIG. 1, according to various embodiments. As shown, the medical device 100 includes an awl 202, a cannula 204, a display 802, and wires 402. The awl 202 includes a distal portion 206 and a handle 208. The cannula 204 includes a distal portion 212 and a handle 214.

As shown, two or more electrodes 210 are disposed on the distal portion 206 of the awl 202. While not shown, the two or more electrodes 210 disposed on the distal portion 206 of the awl 202 are coupled to external electrical components 106 by the wires 402. The external electrical components 106 can generate current at one or more frequencies that is conducted between at least two of the two or more electrodes 210. The external electrical components 106 can generate current at one or more frequencies that is conducted between at least two electrodes 210 of the two or more electrodes 210. The external electrical components 106 can record impedance measurements 708 while the current is conducted between at least two of the two or more electrodes 210. Based on the impedance measurements 708, the processor 706 can determine one or more tissue types 712 of tissue at the location 102 associated with the distal portion 206 of the awl 202. The processor 706 can generate and present, on the display 802, a graphical representation 804 of the impedance measurements 708. In various embodiments, the graphical representation 804 includes a chart that depicts at least one impedance measurement of tissue at different locations 102 associated with the distal portion 206 of the awl 202, such as (without limitation) different depths within the body of the patient, on different sides of the trocar 104, and/or at different times during the surgical procedure. In various embodiments, the graphical representation 804 includes a chart that depicts a determined tissue type 712 of tissue at different locations 102 associated with the distal portion 206 of the awl 202 such as (without limitation) different depths within the body of the patient, on different sides of the trocar 104, and/or at different times during the surgical procedure. In various embodiments, the graphical representation 804 includes a chart that depicts a probability distribution of tissue types 712 of tissue at one or more locations 102 associated with the distal portion 206 of the awl 202, such as (without limitation) different depths within the body of the patient, on different sides of the trocar 104, and/or at different times during the surgical procedure. In various embodiments, the graphical representation 804 includes a chart that depicts one or more operations of a component included in a distal portion 600 of the trocar 104, such as an activation of a drug delivery tool, an energy delivery tool, a tissue sample extraction tool, a pump, or a camera. In various embodiments, the graphical representation 804 includes one or more trocar navigation instructions in order to navigate the distal portion 212 of the awl 202 toward a location 102 of the surgical procedure. For example (without limitation), the one or more trocar navigation instructions can include an instruction to insert the trocar 104 further into the tissue, to withdraw the trocar 104 from the tissue, and/or to maneuver the trocar 104 in a lateral direction.

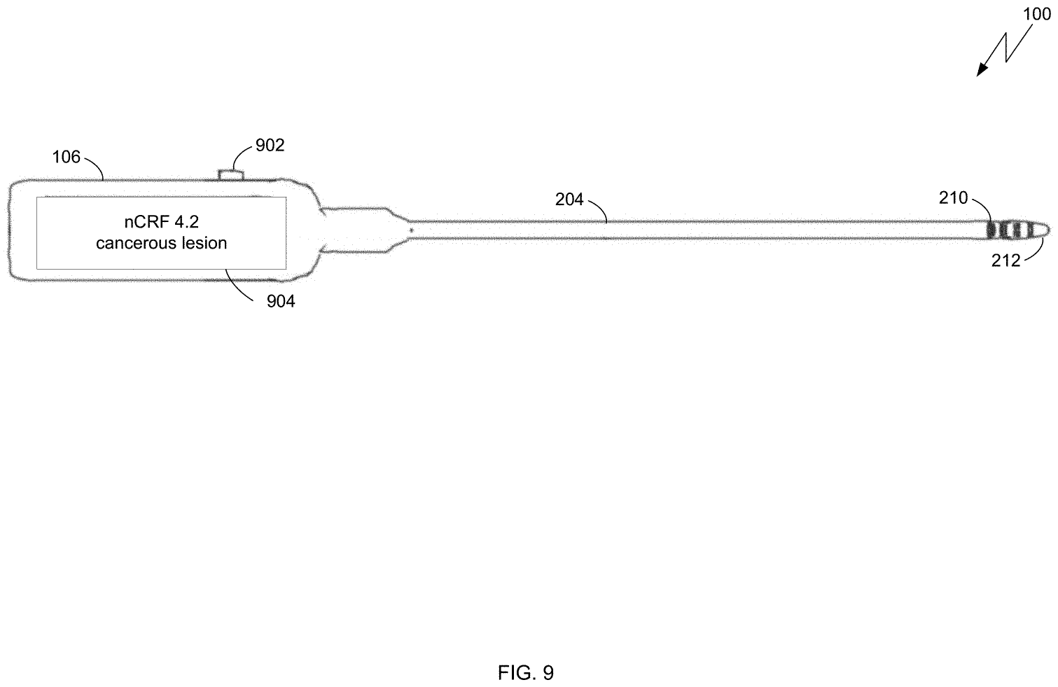

FIG. 9 is a more detailed illustration of the medical device 100 of FIG. 1, according to other various embodiments. As shown, the medical device 100 includes a cannula 204 including a distal portion 212 and external electrical components 106. The distal portion 212 of the cannula 204 includes two or more electrodes 210. The external electrical components 106 include, without limitation, an activation button 902 and a display 904.

While not shown, the medical device 100 includes an awl 202 that is inserted into a body of a patient. The cannula 204 slides down the length of the awl 202 and the distal portion 212 is positioned at the location of the surgical procedure. The external electrical components 106 generate current at various frequencies. Wires within the cannula 204 conduct the current between the external electrical components 106 and the two or more electrodes 210 disposed on the distal portion 212 of the cannula 204. The external electrical components 106 include a processor 706 that couples to the two or more electrodes 210 disposed on the distal portion 212 of the cannula 204. In response to an activation associated with the activation button 902, the processor 706 measures the impedance of current conducted through tissue between at least two of the two or more electrodes 210. Based on the impedance measurements, one or more tissue types at the location 102 associated with the distal portion 212 of the cannula 204. For example and without limitation, based on the impedance measurements, the tissue type can indicate whether tissue at the location 102 associated with the distal portion 212 of the cannula 204 is a tumor or non-tumor. For example and without limitation, based on the impedance measurements, the tissue type can indicate whether tissue at the location 102 associated with the distal portion 212 of the cannula 204 is a cyst or a non-cyst. As shown, the medical device 100 displays the tissue type on the display 904. In some embodiments and as shown, the medical device 100 also displays one or more properties associated with the impedance measurements, such as a normalized Cole relaxation function (“nCRF”) measurement.

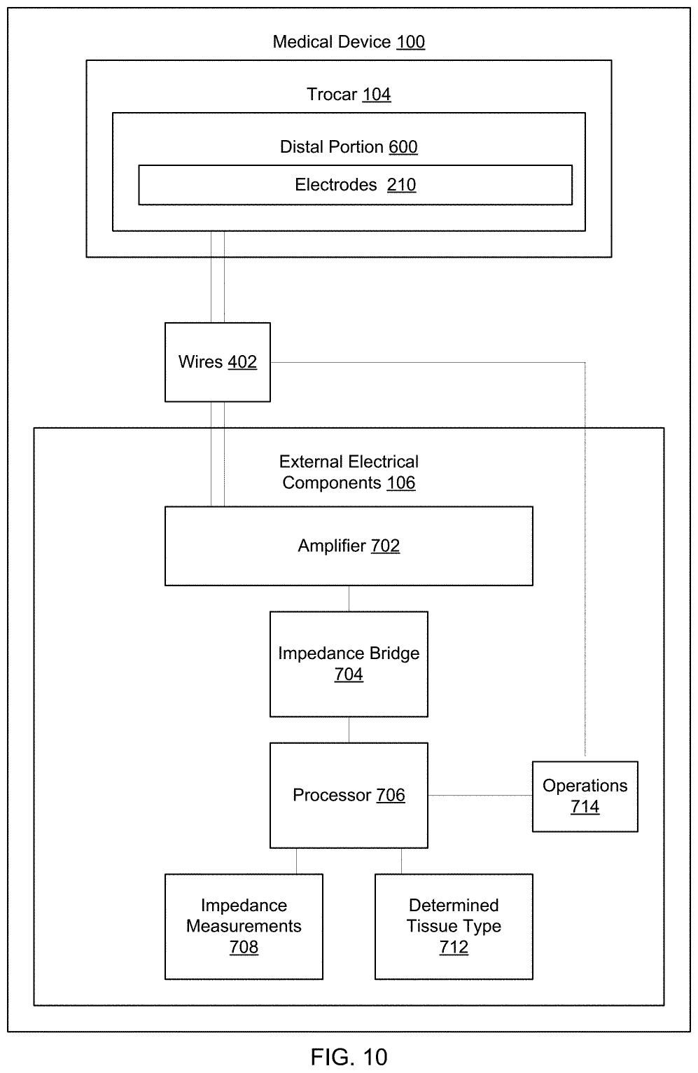

FIG. 10 is a more detailed illustration of the medical device of FIG. 2, according to various embodiments. As shown, the medical device 100 includes a trocar 104, external electrical components 106, and wires 402. As shown, the trocar 104 108 includes a distal portion 600, and the distal portion 600 includes two or more electrodes 210 that are disposed on the distal portion 600. In various embodiments, the two or more electrodes 210 are disposed on a distal portion 206 of an awl 202 and/or a distal portion 212 of a cannula 204. In some embodiments, the distal portion 600 includes a component, such as and without limitation, a therapeutic drug delivery tool, an energy delivery tool, a tissue sample extraction tool, a pump, or a camera. In various embodiments, the distal portion 600 includes, without limitation, two or more components, which can be of one kind or of different kinds.

As shown, the wires 402 couple the two or more electrodes 210 to the external electrical components 106. In various embodiments, without limitation, each of the two or more electrodes 210 is coupled to the external electrical components 106 by one wire 402 or by respective wires of a plurality of wires 402.

As shown, the external electrical components 106 include an amplifier 702, an impedance bridge 704, and a processor 706. The amplifier 702 amplifies a supplied voltage and/or a return voltage while the wires 402 conduct current at various frequencies between the impedance bridge 704 and the two or more electrodes 210. The impedance bridge 704 is an impedance load that the processor 706 measures to determine an impedance of a circuit including the impedance bridge 704, the amplifier 702, the wires 402, and the two or more electrodes 210. The processor 706 records, at various frequencies, one or more impedance measurements 708. The processor 706 compares the two or more impedance measurements 708 with characteristic impedance measurements 710 of respective one or more tissue types. Based on the one or more impedance measurements 708 with characteristic tissue types 712, the processor 706 determines one or more tissue types 712 at the location 102 associated with the distal portion 600 of the trocar 104. In various embodiments and without limitation, the processor 706 determines the tissue type 712 indicated by the respective impedance measurements 708 based on a Cole relaxation frequency of a portion of tissue contacting the two or more electrodes 210. In various embodiments and without limitation, the processor 706 determines the tissue type 712 as areas of tumor tissue types and/or non-tumor tissue types. In various embodiments and without limitation, the processor 706 determines the tissue type 712 as cyst tissue types and/or non-cyst tissue types.

As shown, based on the one or more tissue types 712 at the location 102 associated with the distal portion 600 of the trocar 104, the processor 706 performs one or more operations 714. In various embodiments and without limitation, if the tissue type determined at the location 102 associated with the distal portion 600 of the trocar 104 is a tumor tissue type, the processor 706 performs an operation 714 of outputting a visual indication or an audio indication of the determined tissue type. In various embodiments and without limitation, the medical device 100 reports the one or more tissue types 712 at the location 102 associated with the distal portion 600 of the trocar 104 to a user of the medical device 100. For example and without limitation, the medical device 100 can display the one or more tissue types 712 at the location 102 associated with the distal portion 600 of the trocar 104 using a visual output (e.g., without limitation, a liquid crystal display (LCD), a light-emitting diode (LED) display to present a visual indication of the one or more tissue types 712 at the location 102 associated with the distal portion 600 of the trocar 104, such as a light, symbol, text, graphic, or the like). In various embodiments and without limitation, the visual indication can indicate the one or more tissue types 712 at the location 102 associated with the distal portion 600 of the trocar 104 is a cyst tissue type. The visual indication can indicate whether or not the distal portion 600 of the trocar 104 is positioned at the location 102 of the surgical procedure. The visual indication can indicate whether the tissue type at the location 102 associated with the distal portion 600 of the trocar 104 is a tumor tissue type or a non-tumor tissue type. The visual indication can indicate whether the tissue type at the location 102 associated with the distal portion 600 of the trocar 104 is a cyst tissue type or a non-cyst tissue type.

Alternatively or additionally, in various embodiments, the processor 706 performs one or more operations 714 associated with additional components associated with the distal portion 600 of the trocar 104. In various embodiments and without limitation, the distal portion 600 of the trocar 104 includes a therapeutic drug delivery tool. If the tissue type determined at the location 102 associated with the distal portion 600 of the trocar 104 is a tumor tissue type or a cyst tissue type, the processor 706 performs an operation 714 of causing the therapeutic drug delivery tool to deliver one or more therapeutic drugs to tissue at the location 102 associated with the distal portion 600 of the trocar 104. For example and without limitation, the processor 706 can cause one or more therapeutic drugs through one or more drug delivery conduits to and through the distal portion 600 of the trocar 104.

In various embodiments and without limitation, the distal portion 600 of the trocar 104 includes an energy delivery tool. If the tissue type determined at the location 102 associated with the distal portion 600 of the trocar 104 is a tumor tissue type or a cyst tissue type, the processor 706 performs an operation 714 of causing the energy delivery tool to deliver energy to tissue at the location 102 associated with the distal portion 600 of the trocar 104. For example and without limitation, the processor 706 can cause current to be conducted through the wires 1402 to and through the distal portion 600 of the trocar 104.

In various embodiments and without limitation, the distal portion 600 of the trocar 104 includes a tissue sample extraction tool. If the tissue type determined at the location 102 associated with the distal portion 600 of the trocar 104 is a tumor tissue type or a cyst tissue type, the processor 706 performs an operation 714 of causing the tissue sample extraction tool to extract a sample of tissue at the location 102 associated with the distal portion 600 of the trocar 104. For example and without limitation, the processor 706 can cause the tissue sample extraction tool to cut a sample of the tissue at the location 102 associated with the distal portion 600 of the trocar 104 and to capture the sample of the tissue for extraction and inspection.

In various embodiments and without limitation, the distal portion 600 of the trocar 104 includes a pump. If the tissue type determined at the location 102 associated with the distal portion 600 of the trocar 104 is a tumor tissue type or a cyst tissue type, the processor 706 performs an operation 714 of activating the pump. For example and without limitation, the processor 706 can cause the pump to create a vacuum to drain fluid and/or gas from tissue at the location 102 associated with the distal portion 600 of the trocar 104, such as a tumor or a cyst.

In various embodiments and without limitation, the distal portion 600 of the trocar 104 includes a camera. If the tissue type determined at the location 102 associated with the distal portion 600 of the trocar 104 is a tumor tissue type or a cyst tissue type, the processor 706 performs an operation 714 of activating the camera to capture one or more images of tissue at the location 102 associated with the distal portion 600 of the trocar 104. The one or more images can be shown to a healthcare professional during the surgical procedure. The one or more images can be added to a healthcare record of the patient that documents of the surgical procedure.

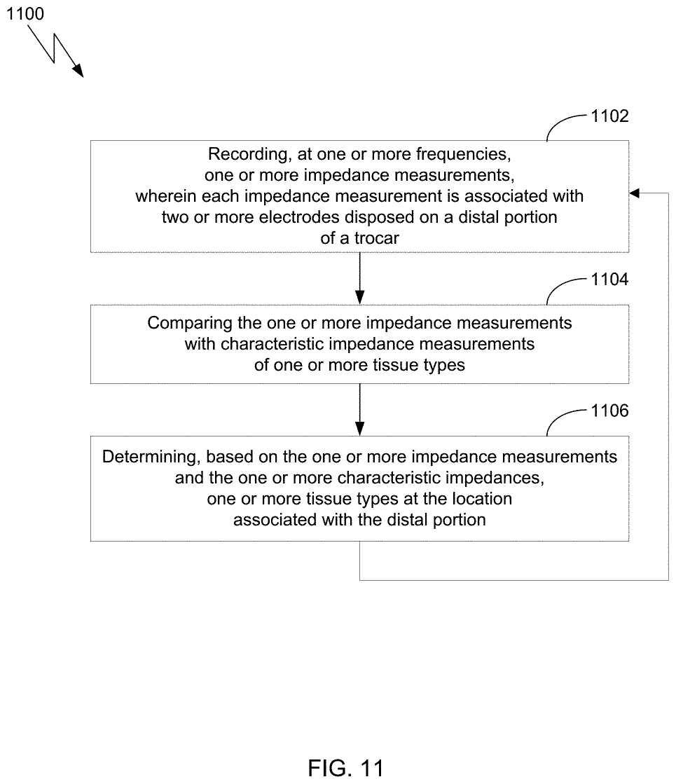

FIG. 11 is a flow diagram of method steps for determining one or more tissue types at a location associated with a distal portion of a trocar, according to various embodiments. Although the method steps are described in conjunction with the systems of FIGS. 1-10, persons skilled in the art will understand that any system configured to perform the method steps, in any order, falls within the scope of the present invention.

As shown, a method 1100 begins at step 1102, where a processor records, at one or more frequencies, one or more impedance measurements, wherein each impedance measurement is associated with two or more electrodes disposed on a distal portion of a trocar. In various embodiments and without limitation, the processor determines a Cole relaxation frequency of tissue between at least two of the two or more electrodes disposed on the distal portion of the trocar (e.g., without limitation, as a frequency of a maximum normalized impedance measurement of the tissue between at least two of the two or more electrodes).

At step 1104, the processor compares the one or more impedance measurements to one or more characteristic impedances associated with one or more tissue types. As an example and without limitation, the processor can compare the impedance measurements with a first set of one or more characteristic impedance measurements of a non-tumor tissue type and a second set of one or more characteristic impedance measurements of a tumor tissue type. As another example and without limitation, the processor can compare the impedance measurements with a first set of one or more characteristic impedance measurements of a non-cyst tissue type and a second set of one or more characteristic impedance measurements of a cyst tissue type.

At step 1106, the processor determines, based on the one or more impedance measurements and the one or more characteristic impedances, one or more tissue types at a location associated with the distal portion. In various embodiments and without limitation, the processor determines the tissue type that classifies the tissue as one of a tumor tissue type or a non-tumor tissue type. Alternatively or additionally, in various embodiments and without limitation, the processor determines the tissue type that classifies the tissue as one of a cyst tissue type or a non-cyst tissue type. In various embodiments and without limitation, the processor determines whether the tissue type at the location associated with the distal portion of the trocar matches an expected tissue type at the location of the surgical procedure. The method can return to step 1102 to record additional impedance measurements and to determine a second or updated tissue type.

In sum, the disclosed medical device records, at one or more frequencies, one or more impedance measurements associated with two or more electrodes disposed on the distal portion of the trocar. The medical device determines the tissue type of tissue in a location associated with a distal portion of a trocar based on the one or more impedance measurements. The disclosed approach advantageously results in the medical device determining the tissue type of tissue associated with the location associated with the distal portion of the trocar.

At least one technical advantage of the disclosed medical device relative to the prior art is that the disclosed medical device is able to automatically and accurately determine the tissue type at a location associated with the distal portion of the trocar during a surgical procedure. For example, the disclosed trocar can accurately determine whether the tissue type at a location associated with the distal portion of the trocar is a tumor tissue type, a cyst tissue type, or a non-tumor and non-cyst tissue type. Determining the tissue type at the location associated with the distal portion of the trocar with high accuracy can advantageously reduce the incidence of false positive testing outcomes and reduce the occurrence of unnecessary medical procedures. Determining the tissue type of the tissue associated with the distal portion of the trocar with high accuracy also can advantageously reduce the incidence of false negative testing outcomes and enable effective cancer treatment at early stages and with better prognoses. In addition, the disclosed medical device can automatically and accurately determine whether the tissue type at the location associated with the distal portion of the trocar matches an expected tissue type at the location of the surgical procedure. Such determinations can improve overall confidence in the test results. Likewise, automatically and accurately determining that the tissue type at a location associated distal portion of the trocar does not match the expected tissue type can reduce incorrect test results and give a healthcare professional an opportunity to reposition the trocar. As yet another technical advantage, the disclosed medical device enables the trocar to serve as a diagnostic probe that determines the tissue type in addition the other uses of the trocar, such as providing a passage for other medical instruments. Because the trocar serves as a diagnostic probe, the healthcare professional does not need to introduce a separate diagnostic probe through the cannula of the trocar, reducing the number of instruments at the location associated with the distal portion of the trocar. These technical advantages provide one or more technological advancements over prior art designs and approaches.

[Claim Combinations to be Added Prior to Filing]

Any and all combinations of any of the claim elements recited in any of the claims and/or any elements described in this application, in any fashion, fall within the contemplated scope of the present invention and protection.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

Aspects of the present embodiments may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “module,” a “system,” or a “computer.” In addition, any hardware and/or software technique, process, function, component, engine, module, or system described in the present disclosure may be implemented as a circuit or set of circuits. Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine. The instructions, when executed via the processor of the computer or other programmable data processing apparatus, enable the implementation of the functions/acts specified in the flowchart and/or block diagram block or blocks. Such processors may be, without limitation, general purpose processors, special-purpose processors, application-specific processors, or field-programmable gate arrays.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the preceding is directed to embodiments of the present disclosure, other and further embodiments of the disclosure may be devised without departing from the basic scope thereof, and the scope thereof is determined by the claims that follow.

What is claimed is:

1. A medical device, comprising:
a trocar including an awl and a cannula;
two or more electrodes disposed on a distal portion of the trocar;
an impedance bridge coupled to the two or more electrodes; and
a processor coupled to the impedance bridge.

2. The medical device of claim 1, wherein the two or more electrodes are disposed on an outer surface of the awl.

3. The medical device of claim 1, wherein the two or more electrodes are disposed on an outer surface of the cannula.

4. The medical device of claim 1, wherein the two or more electrodes are disposed on an inner surface of the cannula.

5. The medical device of claim 1 wherein at least one of the two or more electrodes is disposed on a surface of the awl, and at least another one of the two or more electrodes is disposed on a surface of the cannula.

6. The medical device of claim 1, wherein each of the two or more electrodes is located at a respective location along a length of the trocar.

7. The medical device of claim 1, wherein at least one of the two or more electrodes is located on a first side of the trocar, and at least another one of the two or more electrodes is located on a second side of the trocar.

8. The medical device of claim 1, further comprising one or more wires that couple the two or more electrodes to the impedance bridge, wherein at least a portion of the one or more wires is imprinted on a surface of the trocar.

9. The medical device of claim 1, further comprising a display coupled to the processor.

10. The medical device of claim 1, further comprising a wireless transmitter coupled to the processor.

11. The medical device of claim 1, wherein the processor selectively couples the impedance bridge to at least two electrodes of the two or more electrodes.

12. The medical device of claim 1, further comprising a component coupled to the cannula, wherein the component includes at least one of a therapeutic drug delivery tool, an energy delivery tool, a tissue sample extraction tool, a pump, or a camera.

13. A computer-implemented method for evaluating tissue of a patient, the method comprising:
recording, at one or more frequencies, one or more impedance measurements, wherein each impedance measurement is associated with two or more electrodes disposed on a distal portion of a trocar;
comparing the one or more impedance measurements to one or more characteristic impedances associated with one or more tissue types; and
determining, based on the one or more impedance measurements and the one or more characteristic impedances, one or more tissue types at a location associated with the distal portion of the trocar.

14. The computer-implemented method of claim 13, wherein the one or more impedance measurements are recorded in response to an activation associated with an activation button.

15. The computer-implemented method of claim 13, wherein the one or more impedance measurements include a first impedance measurement associated with a first location of a cannula along a length of an awl and a second impedance measurement associated with a second location of the cannula along the length of the awl.

16. The computer-implemented method of claim 13, wherein the one or more impedance measurements include a first impedance measurement associated with a first subset of the two or more electrodes and a second impedance measurement associated with a second subset of the two or more electrodes.

17. The computer-implemented method of claim 16, wherein the first subset of the two or more electrodes is associated with a first location along the distal portion of the trocar, and the second subset of the two or more electrodes is associated with a second location along the distal portion of the trocar.

18. The computer-implemented method of claim 16, wherein the first subset of the two or more electrodes is associated with a first side of the distal portion of the trocar, and the second subset of the two or more electrodes is associated with a second side of the distal portion of the trocar.

19. The computer-implemented method of claim 13, further comprising measuring one or more dimensions of tissue at the location associated with the distal portion of the trocar.

20. The computer-implemented method of claim 13, further comprising outputting at least one of a visual indication or an audio indication of the one or more tissue types at the location associated with the distal portion of the trocar.

21. The computer-implemented method of claim 13, further comprising outputting at least one trocar navigation instruction based on the one or more tissue types at the location associated with the distal portion of the trocar.

22. The computer-implemented method of claim 13, further comprising determining a confidence score of the one or more tissue types at the location associated with the distal portion of the trocar.

23. The computer-implemented method of claim 13, further comprising transmitting, using a wireless transmitter, a wireless signal indicating the one or more tissue types at the location associated with the distal portion of the trocar.

24. The computer-implemented method of claim 13, further comprising activating a component coupled to a distal portion of a cannula based on the one or more tissue types at the location associated with the distal portion of the trocar, wherein the component includes at least one of a therapeutic drug delivery tool, an energy delivery tool, a tissue sample extraction tool, pump, or a camera.
